(19) United States
(12) Patent Application Publication
(10) Pub. No.: US 2010/0152676 A1
(43) Pub. Date: Jun. 17, 2010

(54) Drug Loading Through Plunger

(75) Inventors: Don A. Clements, Cleburne, TX (US); Julie A. Clifford, Arlington, TX (US); Bruno Dacquay, Irvine, CA (US); Mark T. DuPriest, Hudson Oaks, TX (US); Robert Rodstrom, Cransfills Gap, TX (US); Robert J. Sanchez, JR., Oceanside, CA (US); Pardeep K. Sethi, Fort Worth, TX (US)

Correspondence Address: ALCON, IP LEGAL, TB4-8, 6201 SOUTH FREEWAY, FORT WORTH, TX, 76134, US

(73) Assignee: Alcon Research, LTD., Fort Worth, TX (US)

(21) Appl. No.: 12/635,334

(22) Filed: Dec. 10, 2009

Related U.S. Application Data
(60) Provisional application No. 61/122,864, filed on Dec. 16, 2008.

(51) Int. Cl. A61M 5/315; B65B 3/04

(52) U.S. Cl. 604/231; 141/2

(57) ABSTRACT

A drug loading apparatus includes a plunger with a through hole or slit valve located in the top face of the plunger. A dispensing chamber housing located in a drug delivery device is configured to receive the plunger so that a dispensing chamber is formed when the plunger is located in the dispensing chamber housing. The drug can then be loaded into the dispensing chamber through the plunger, and a piston with an end configured to seal the through hole or engage the plunger with the slit valve can then be inserted.

### BACKGROUND OF THE INVENTION

This application claims the benefit of U.S. Provisional Patent Application No. 61/122,864 filed Dec. 16, 2008.

The present invention relates to loading a drug into an injection device and more particularly to an apparatus and system for loading a drug through the plunger of an injection device.

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

These, and other diseases, can be treated by injecting a drug into the eye. Such injections are typically done manually using a conventional syringe and needle. FIG. 1 is a perspective view of a prior art syringe used to inject drugs into the eye. In FIG. 1, the syringe includes a needle 105, a luer hub 110, a chamber 115, a plunger 120, a plunger shaft 125, and a thumb rest 130. As is commonly known, the drug to be injected is located in chamber 115. Pushing on the thumb rest 130 causes the plunger 120 to expel the drug through needle 105.

In using such a syringe, the surgeon is required to pierce the eye tissue with the needle, hold the syringe steady, and actuate the syringe plunger (with or without assistance) to inject the fluid into the eye. Fluid flow rates are uncontrolled. The volume injected is typically not controlled in an accurate manner because reading the vernier is subject to parallax error. Tissue damage may occur due to an “unsteady” injection.

An effort has been made to control the delivery of small amounts of liquids. A commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. Ypsomed, Inc. of Switzerland produces a line of injection pens and automated injectors primarily for the self-injection of insulin or hormones by a patient. This product line includes simple disposable pens and electronically-controlled motorized injectors.

U.S. Pat. No. 6,290,690 discloses an ophthalmic system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

When a portable hand piece is used to inject a drug into the eye, it is important to provide a proper drug dosage. In one case, a phase transition compound or reverse gelation compound contains the drug. At room temperature, these compounds are in a solid state and have the consistency of wax. Because of their consistency, dosing an injector with these compounds can be difficult. The compounds can be brought to a more liquid state and drawn into the injector. However, this is a time consuming process that may not provide proper dosage. It would be desirable to have a system for accurately and quickly loading such a drug mixture into an injection device.

### SUMMARY OF THE INVENTION

In one embodiment consistent with the principles of the present invention, the present invention is a drug loading apparatus. The apparatus includes a plunger with a through hole located in the top face of the plunger, a dispensing chamber housing located in a drug delivery device, the dispensing chamber housing configured to receive the plunger so that a dispensing chamber is formed when the plunger is located in the dispensing chamber housing, and a piston with an end configured to seal the through hole. Alternatively, instead of a through hole, a slit valve is located in the top face of the plunger. The piston then engages the slit valve to the closed position within the plunger.

In another embodiment consistent with the principles of the present invention, the present invention is a method of loading an ophthalmic injection device including placing a plunger in a dispensing chamber housing to form a dispensing chamber when the plunger is located in the dispensing chamber housing; inserting a drug loading mechanism into the plunger; and filling the dispensing chamber through the plunger.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying figures, which are incorporated in and constitute a part of this specification, illustrate several embodiments of the invention and together with the description, serve to explain the principles of the invention.

FIG. 1 is a perspective view of a prior art syringe.

FIG. 2 is a cross section view of a disposable tip segment and a limited reuse assembly according to the principles of the present invention.

FIGS. 3A and 3B are cross section and top views, respectively, of a plunger with a through hole for drug loading according to the principles of the present invention.

FIGS. 4A, 4B, and 4C are cross section and top views, respectively, of a plunger with a through hole for drug loading according to the principles of the present invention.

FIGS. 5A and 5B are cross section and top views, respectively, of a plunger with a slit valve for drug loading according to the principles of the present invention.

FIGS. 6A-6E are cross section views of a system for loading a drug through a plunger according to the principles of the present invention.

FIG. 7 is a perspective view of a plunger and piston assembly according to the principles of the present invention.

FIGS. 8A and 8B are cross section views of a plunger with a through hole and a piston according to the principles of the present invention.

FIGS. 9A and 9B are cross section and top views, respectively, of a plunger with a through hole for drug loading according to the principles of the present invention.

FIGS. 10A-10C are cross section views of a system for loading a drug through a plunger according to the principles of the present invention.

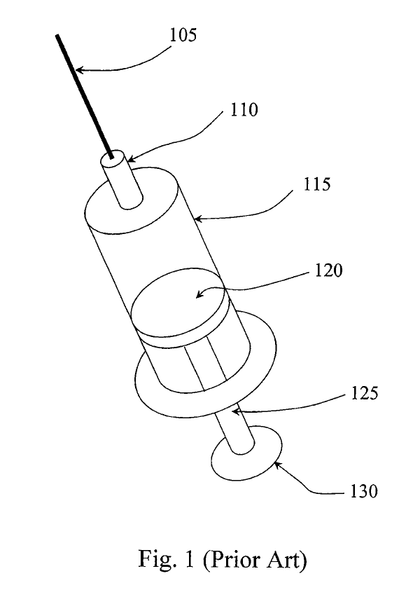

### DETAILED DESCRIPTION OF THE PREFERRED EMBODIMENTS

Reference is now made in detail to the exemplary embodiments of the invention, examples of which are illustrated in the accompanying figures. Wherever possible, the same reference numbers are used throughout the figures to refer to the same or like parts.

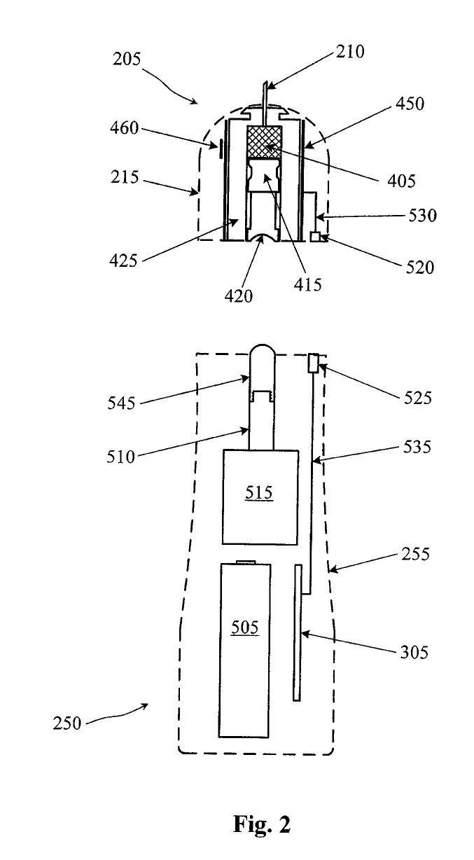

FIG. 2 is a cross section view of a disposable tip segment and a limited reuse assembly according to an embodiment of the present invention. FIG. 2 shows how tip segment 205 interfaces with limited reuse assembly 250. In the embodiment of FIG. 2, tip segment 205 includes plunger interface 420, plunger 415, dispensing chamber housing 425, tip segment housing 215, temperature control device 450, thermal sensor 460, needle 210, dispensing chamber 405, interface 530, and tip interface connector 520. Limited reuse assembly 250 includes mechanical linkage 545, actuator shaft 510, actuator 515, power source 505, controller 305, limited reuse assembly housing 255, interface 535, and limited reuse assembly interface connector 525.

In tip segment 205, plunger interface 420 is located on one end of plunger 415. The other end of plunger 415 forms one end of dispensing chamber 405. Plunger 415 is adapted to slide within dispensing chamber 405. An outer surface of plunger 415 is fluidly sealed to the inner surface of dispensing chamber housing 425. Dispensing chamber housing 425 surrounds the dispensing chamber 405. Typically, dispensing chamber housing 425 has a cylindrical shape. As such, dispensing chamber 405 also has a cylindrical shape.

Needle 210 is fluidly coupled to dispensing chamber 405. In such a case, a substance contained in dispensing chamber 405 can pass through needle 210 and into an eye. Temperature control device 450 at least partially surrounds dispensing chamber housing 425. In this case, temperature control device 450 is adapted to heat and/or cool dispensing chamber housing 425 and any substance contained in dispensing chamber 405. Interface 530 connects temperature control device 450 and thermal sensor 460 with tip interface connector 520.

The components of tip segment 205, including dispensing chamber housing 425, temperature control device 450, and plunger 415 are at least partially enclosed by tip segment housing 215. In one embodiment consistent with the principles of the present invention, plunger 415 is sealed to the interior surface of dispensing chamber housing 425. This seal prevents contamination of any substance contained in dispensing chamber 405. For medical purposes, such a seal is desirable. This seal can be located at any point on plunger 415 or dispensing chamber housing 425.

In limited reuse assembly 250, power source 505 provides power to actuator 515. An interface (not shown) between power source 505 and actuator 515 serves as a conduit for providing power to actuator 515. Actuator 515 is connected to actuator shaft 510. When actuator 515 is a stepper motor, actuator shaft 510 is integral with actuator 515. Mechanical linkage interface 545 is connected to actuator shaft 510. In this configuration, as actuator 515 moves actuator shaft 510 upward toward needle 210 mechanical linkage interface 545 also moves upward toward needle 210.

Controller 305 is connected via interface 535 to limited reuse assembly interface connecter 525. Limited reuse assembly interface connecter 525 is located on a top surface of limited reuse assembly housing 255 adjacent to mechanical linkage interface 545. In this manner, both limited reuse assembly interface connector 525 and mechanical linkage interface 545 are adapted to be connected with tip interface connector 520 and plunger interface 420 respectively.

Controller 305 and actuator 515 are connected by an interface (not shown). This interface (not shown) allows controller 305 to control the operation of actuator 515. In addition, an interface (not shown) between power source 505 and controller 305 allows controller 305 to control operation of power source of 310. In such a case, controller 305 may control the charging and the discharging of power source 505 when power source 505 is a rechargeable battery.

Controller 305 is typically an integrated circuit with power, input, and output pins capable of performing logic functions. In various embodiments, controller 305 is a targeted device controller. In such a case, controller 305 performs specific control functions targeted to a specific device or component, such as a temperature control device or a power supply. For example, a temperature control device controller has the basic functionality to control a temperature control device. In other embodiments, controller 305 is a microprocessor. In such a case, controller 305 is programmable so that it can function to control more than one component of the device. In other cases, controller 305 is not a programmable microprocessor, but instead is a special purpose controller configured to control different components that perform different functions. While depicted as one component, controller 305 may be made of many different components or integrated circuits.

Tip segment 205 is adapted to mate with or attach to limited reuse assembly 250 as previously described. In the embodiment of FIG. 5, plunger interface 420 located on a bottom surface of plunger 415 is adapted to mate with mechanical linkage interface 545 located near a top surface of limited reuse assembly housing 255. In addition, tip interface connector 520 is adapted to connect with limited reuse assembly interface connector 525. When tip segment 205 is connected to limited reuse assembly 250 in this manner, actuator 515 and actuator shaft 510 are adapted to drive plunger 415 upward toward needle 210. In addition, an interface is formed between controller 305 and temperature control device 450. A signal can pass from controller 305 to temperature control device 450 through interface 535, limited reuse assembly interface connector 525, tip interface connector 520, and interface 530.

In operation, when tip segment 205 is connected to limited reuse assembly 250, controller 305 controls the operation of actuator 515. Actuator 515 is actuated and actuator shaft 510 is moved upward toward needle 210. In turn, mechanical linkage interface 545, which is mated with plunger interface 420, moves plunger 415 upward toward needle 210. A substance located in dispensing chamber 405 is then expelled through needle 210.

In addition, controller 305 controls the operation of temperature control device 450. Temperature control device 450 is adapted to heat and/or cool dispensing chamber housing 425. Since dispensing chamber housing 425 is at least partially thermally conductive, heating or cooling dispensing chamber housing 425 heats or cools a substance located in dispensing chamber 405. Temperature information can be transferred from thermal sensor 460 to controller 305 via any of a number of different interface configurations. This temperature information can be used to control the operation of temperature control device 450. When temperature control device 450 is a heater, controller 305 controls the amount of current that is sent to temperature control device 450. The more current sent to temperature control device 450, the hotter it gets. In such a manner, controller 305 can use a feed back loop utilizing information from thermal sensor 460 to control the operation of temperature control device 450. Any suitable type of control algorithm, such as a proportional integral derivative (PID) algorithm, can be used to control the operation of temperature control device 450.

In various embodiments of the present invention, temperature control device 450 heats a phase transition compound that is located in dispensing chamber 405. This phase transition compound carries a drug that is to be injected into the eye. A phase transition compound is in a solid or semi-solid state at lower temperatures and in a more liquid state at higher temperatures. Such a substance can be heated by temperature control device 450 to a more liquid state and injected into the eye where it forms a bolus that erodes over time. Likewise, a reverse gelation compound may be used. A reverse gelation compound is in a solid or semi-solid state at higher temperatures and in a more liquid state at lower temperatures. Such a compound can be cooled by temperature control device 450 to a more liquid state and injected into the eye where it forms a bolus that erodes over time. As such, temperature control device 450 may be a device that heats a substance in dispensing chamber 405 or a device that cools a substance in dispensing chamber 405 (or a combination of both). After being delivered into the eye, a phase transition compound or reverse gelation compound erodes over time providing a quantity of drug over an extended period of time. Using a phase transition compound or reverse gelation compound provides better drug dosage with fewer injections.

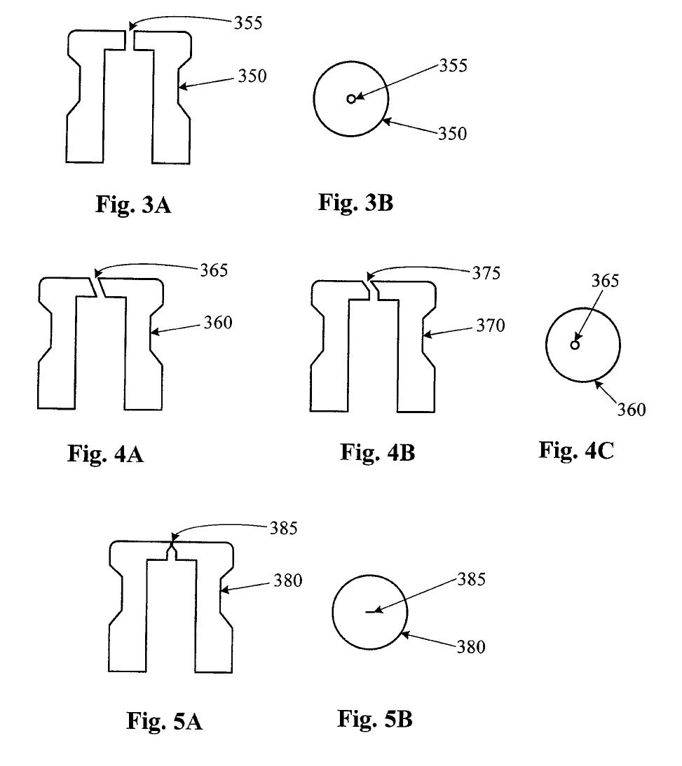

FIGS. 3A and 3B are cross section and top views, respectively, of a plunger with a through hole for drug loading according to the principles of the present invention. In FIGS. 3A and 3B, plunger 350 has a through hole 355 in its top surface (the surface that contacts the drug when the plunger is located in the injection device). In this embodiment, through hole 355 is generally round and located at or near the center of the plunger. Through hole 350 is also generally perpendicular to the top surface of plunger 350. While depicted as round, through hole 355 may be of any shape and may be located anywhere on the top surface of plunger 350.

FIGS. 4A, 4B, and 4C are cross section and top views, respectively, of a plunger with a through hole for drug loading according to the principles of the present invention. Plunger 360, 370 has a through hole 365, 375 in its top surface (the surface that contacts the drug when the plunger is located in the injection device). In this embodiment, through hole 365, 375 is generally round and begins at or near the center of the interior of plunger 360, 370 and ends at a point offset from the center of the top face of plunger 360, 370. In this manner, through hole 365, 375 forms a lumen that proceeds at an angle through the top face of plunger 360, 370. In FIG. 4A, this angle is continuous, while in FIG. 4B, this angle is not continuous. In other embodiments, through hole 365, 375 can be at any angle or orientation. While depicted as round, through hole 365, 375 may be of any shape and may be located anywhere on the top surface of plunger 360, 370.

FIGS. 5A and 5B are cross section and top views, respectively, of a plunger with a slit valve for drug loading according to the principles of the present invention. In FIGS. 5A and 5B, plunger 380 has a slit valve 385 in its top surface (the surface that contacts the drug when the plunger is located in the injection device). In this embodiment, slit valve 385 is generally located at or near the center of the plunger. Slit valve 385 is also oriented generally perpendicular to the top surface of plunger 380, although other orientations are also contemplated.

The through holes or slit valve depicted in FIGS. 3A, 3B, 4A, 4B, 4C, 5A, and 5B allow for a drug to be loaded into an injection device through the plunger. In this manner, a disposable section of an injection device that includes a plunger and dispensing chamber can be loaded with the plunger in place. A loading mechanism can inject the drug mixture into the dispensing chamber through the plunger with the plunger in place as described below.

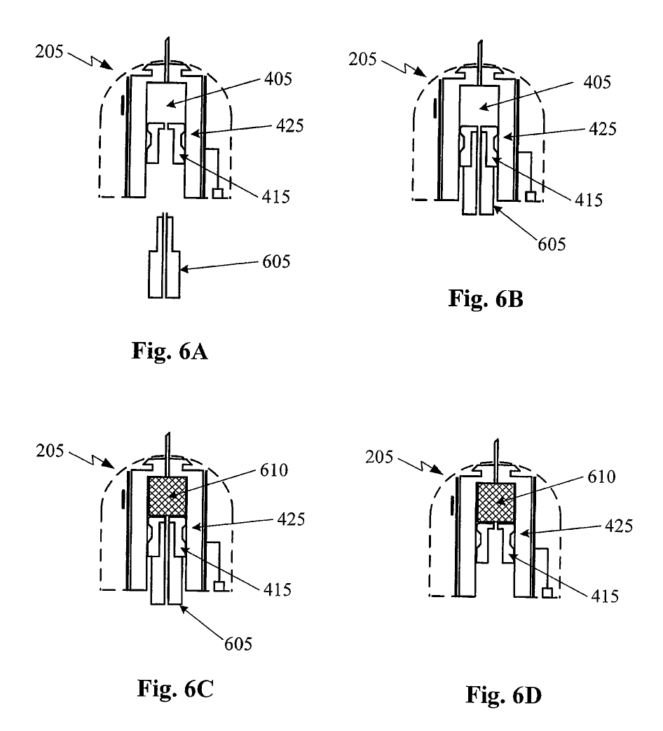

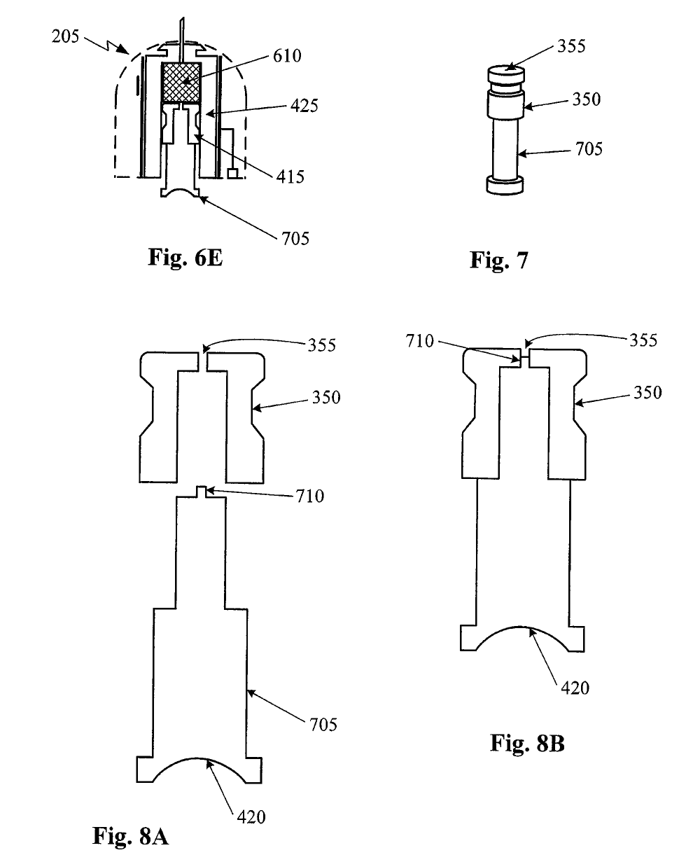

FIGS. 6A-6E are cross section views of a system for loading a drug through a plunger according to the principles of the present invention. In these figures, a loading mechanism 605 is used to inject a drug mixture into dispensing chamber 405 with plunger 415 in place. In FIG. 6A, plunger 415 (depicted with a straight through hole—though other through hole configurations or a slit valve may be employed) is in place—that is plunger 415 is located in dispensing chamber housing 425 so that the top surface of plunger 415 forms one boundary of dispensing chamber 405. Loading mechanism 605 is ready to be inserted into plunger 415.

Loading mechanism 605 has a lumen through it that is used to inject a drug mixture through plunger 415 and into dispensing chamber 405. Loading mechanism 605 may be heated (or temperature controlled as the case may be). If the drug mixture to be injected is a drug/phase transition compound mixture, then it is heated to a more liquid state before being injected into dispensing chamber 405. In this manner, loading mechanism 605 is heated so that the mixture can be properly injected through plunger 415 and into dispensing chamber 405. In addition, dispensing chamber 415 may be heated as well to assist in the drug loading process.

In FIG. 6B, loading mechanism 605 has been inserted into plunger 415. In this position, loading mechanism 605 is ready to inject a mixture into dispensing chamber 405 through plunger 415. In FIG. 6C, loading mechanism 605 has injected the mixture 610 into the dispensing chamber. In FIG. 6D, loading mechanism 605 has been removed. In FIG. 6E, a piston 705 has been inserted into plunger 415. The device is now properly dosed and ready to be shipped. Piston 705 is placed in plunger 415 after loading mechanism 605 has injected the mixture into the dispensing chamber 405 through plunger 415. In this manner, piston 705 acts to plug up the through hole in plunger 415.

FIG. 7 is a perspective view of a plunger and piston assembly according to the principles of the present invention. Piston 705 is placed in plunger 350 to block through hole 355 (as depicted in FIG. 6E).

FIGS. 8A and 8B are cross section views of a plunger with a through hole and a piston according to the principles of the present invention. FIGS. 8A and 8B more clearly show how piston 705 fits into plunger 350 to block through hole 355. A protrusion 710 on the top end of piston 705 fits into through hole 355 to seal it. In this embodiment, through hole 355 has a generally circular cross section and protrusion 710 is generally cylindrical so that it seals through hole 355. In other embodiments, protrusion may have other shapes. For example, when used with the through hole of FIG. 4A (angled through hole), protrusion 710 may be shaped like a cone or a wedge so that protrusion 710 can properly seal the angled through hole.

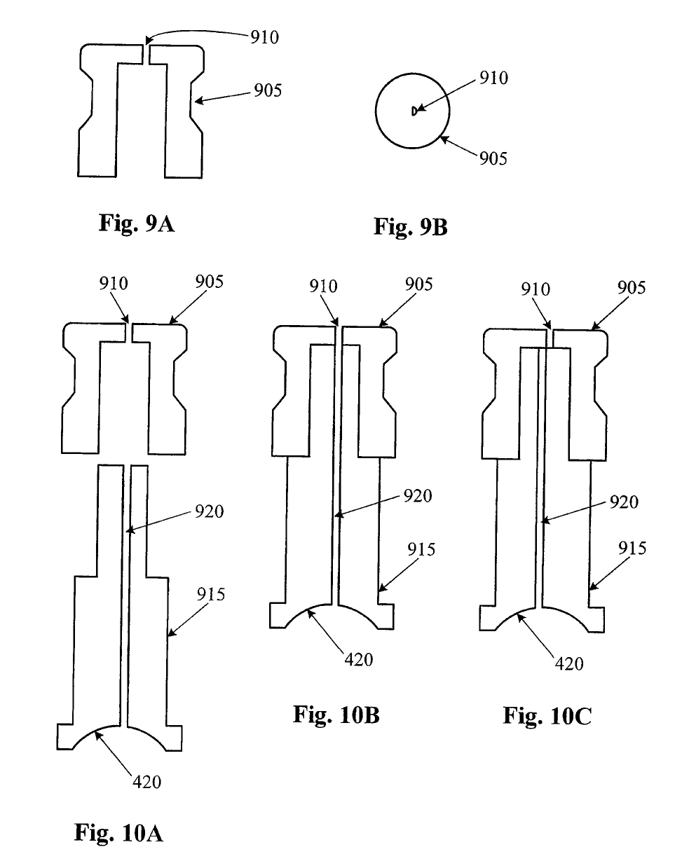

FIGS. 9A and 9B are cross section and top views, respectively, of a plunger with a through hole for drug loading according to the principles of the present invention. In FIGS. 9A and 9B, plunger 905 has a semicircular through hole 910 that is slightly offset from the center of the top face of plunger 905.

FIGS. 10A-10C are cross section views of a system for loading a drug through a plunger according to the principles of the present invention. Piston 915 is generally cylindrical in shape so that it is capable of being rotated when located in plunger 905. Piston 915 has a lumen 920 that has a semicircular cross section. The semicircular lumen 920 is designed to match the semicircular through hole 910 so that a continuous path is formed for the injection of a mixture through plunger 905 (as shown in FIG. 10B). When piston 915 is located in plunger 905 as shown in FIG. 10B, a mixture can be injected through plunger 905 and into a dispensing chamber (as described above). Piston 915 can then be rotated 180 degrees (as shown in FIG. 10C) so that lumen 920 is offset from through hole 910. In this manner, through hole 910 is sealed. In the process shown in FIGS. 10A-10C, the piston itself is used as the drug loading device (and can have its characteristics controlled—for example, it can be heated).

This drug loading process has significant advantages over traditional processes. This process is capable of being automated so that a number of injection devices can be filled. In addition, dosing and placement of the plunger in the device can be much more precise. Loading a drug mixture in a pharmaceutical facility in such a manner also increases the safety of the device—it keeps the mixture in a controlled environment, precisely doses the mixture, and allows for other inspections and quality controls.

From the above, it may be appreciated that the present invention provides an improved system for preparing drug dosage. The present invention provides an apparatus that is designed to reliably make pellets of a consistent quality. This apparatus is configured to form pellets from a drug/compound mixture that is solid at room temperature but liquid at other temperatures. The finished pellets are of the proper size to produce a reliable dosage when injected into the eye.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An apparatus comprising:
a plunger with a through hole located in the top face of the plunger;
a dispensing chamber housing located in a drug delivery device, the dispensing chamber housing configured to receive the plunger so that a dispensing chamber is formed when the plunger is located in the dispensing chamber housing; and
a piston with an end configured to seal the through hole.

2. The apparatus of claim 1 further comprising:
a drug loading apparatus configured to inject a mixture into the dispensing chamber through the plunger.

3. The apparatus of claim 2 wherein the drug loading apparatus is heated.

4. The apparatus of claim 1 further comprising:
a temperature control device at least partially surrounding the dispensing chamber housing.

5. The apparatus of claim 1 wherein the through hole is semicircular and the piston has a semicircular lumen that forms a continuous path for injecting a mixture into the dispensing chamber.

6. The apparatus of claim 5 wherein the piston is capable of being rotated to seal the through hole.

7. The apparatus of claim 1 wherein the through hole is generally perpendicular to a top face of the plunger.

8. The apparatus of claim 1 wherein the through hole is oriented at an angle with respect to a top face of the plunger.

9. The apparatus of claim 1 wherein the end of the piston that is configured to seal the through hole further comprises:
a protrusion that fits into the through hole.

10. A drug loading apparatus comprising:
a plunger with a slit valve located in the top face of the plunger;
a dispensing chamber housing located in a drug delivery device, the dispensing chamber housing configured to receive the plunger so that a dispensing chamber is formed when the plunger is located in the dispensing chamber housing; and
a piston with an end configured to engage the plunger.

11. A method of loading a drug into an injection device comprising:
placing a plunger in a dispensing chamber housing to form a dispensing chamber when the plunger is located in the dispensing chamber housing;
inserting a drug loading mechanism into the plunger; and
filling the dispensing chamber through the plunger.

12. The method of claim 11 further comprising:
removing the drug loading mechanism from the plunger.

13. The method of claim 12 further comprising:
inserting a piston into the plunger after the drug loading mechanism is removed.
